(19) United States
(12) Patent Application Publication
(10) Pub. No.: US 2007/0062541 A1
(43) Pub. Date: Mar. 22, 2007

(54) Device for anatomical support and uses thereof

(76) Inventors: Huan Sue Zhou, Ossining, NY (US); Thomas Ming Chen, Ossining, NY (US); Wu jin Zhou, Bronx, NY (US)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/515,338

(22) Filed: Aug. 31, 2006

Related U.S. Application Data
(60) Provisional application No. 60/713,578, filed on Aug. 31, 2005.

(51) Int. Cl. A61F 6/06

(52) U.S. Cl. 128/830

(57) ABSTRACT

A device for vaginal insertion that corrects the anatomical defect which is the cause of majority of prolapses is disclosed. Said device comprises a base member (or body), two lateral members (or opposite side arms) attached to two opposing sides of said base member, and a protruding member (or handle) attached to the mid-section of said base member. Two lateral arms re-approximate the detached endopelvic fascia to the pelvic side walls at the site of the Arcus Tendineus Levator Ani. In an embodiment, the base member is elongated or rectangularly shaped and/or is concaved toward the vaginal apex. In another embodiment, the base member reduces the prolapsed uterus. The protruding member extends outwardly from the base member to support the bladder neck, thereby improving urinary incontinence. The device is formed of a biocompatible, flexible material, e.g., medical grade silicone, so that it can be folded, for example at a mid-section for insertion.

This application claims priority of U.S. Ser. No. 60/713,578, Filed Aug. 31, 2005. Throughout this application, various publications are referenced. Disclosures of these publications in their entireties are hereby incorporated by reference into this application to more fully describe the state of the art to which this invention pertains.

### BACKGROUND OF THE INVENTION

The present invention relates to the field of medical devices known as pessary for pelvic organ prolapses and urinary incontinence. For pelvic organ prolapse there are currently two methods of treatment: vaginal pessary or surgery. The present available pessaries are generally categorized into three classes: levers, space occupiers and combinations thereof. The levers, such as the Hodge pessary, lift the prolapsed uterus, but generally do not work in severe prolapse. The space occupiers, such as the Ring, Doughnut and Gellhorn push the prolapsed organ up by occupying the vaginal cavity. Examples of these devices can be found in U.S. Pat. Nos. D345,795, 5,611,768, 6,418,930, 5,771,889, 4,307,716, 6,216,698, 5,8947,842 and 6,158,435. None of these pessaries addresses the anatomical defect causing the prolapse. A significant number of patients have prolapse due to detachment of the endopelvic fascia to the Arcus Tendineus Levator Ani, resulting in cystoceles and rectoceles. The device or pessary of the present invention addresses this anatomical condition by, for example, re-approximating the detached endopelvic fascia to the pelvic side walls at the site of the Arcus Tendineus Levator Ani.

Stress urinary incontinence generally results from bladder neck hypermobility. Currently, patients are treated with behavioral therapy, pessaries or surgery. The device or pessary of the present invention comprises an anterior handle which can be configured to support the bladder neck, thereby improving or ameliorating stress incontinence.

The commonly used pessaries, such as the ring shaped pessary, although easy to use, do not work well for more severe form of prolapses. The Gellhorn pessary can be used to treat more severe form of prolapses; however, it is very difficult to manage. It is painful to insert and remove, and patients usually cannot self-manage the Gellhorn pessary.

The device or pessary of the present invention comprises a handle that allows patients to grasp the pessary easily, thus making self management, i.e., removal for cleaning purposes, possible.

The device or pessary of the present invention is also designed for easier insertion and removal. The device or pessary of the present invention is preferably fabricated as a single, integral unit from a resilient, biocompatible material, such as medical grade silicone. The device or pessary of the present invention can also be formed of a softer and/or more pliable material, such as a silicone elastomer, or provided with a softer outer layer. Preferably, the device of the present invention is formed of a material, such as medical grade silicone, suitable for prolonged contact with the human body and capable of maintaining the structural rigidity of the device when placed inside the vaginal cavity of a subject.

The device of the present invention preferably comprises an elongated and parabolic shaped base member, with lateral members on the top surface of said base member extending outwardly from the base member to facilitate the treatment, diagnosis or amelioration of various degrees of pelvic organ prolapse, and a protruding handle on the bottom surface of said base member that allows patients to grasp the pessary easily, and provides additional support to improve or ameliorate stress incontinence.

### OBJECTS OF THE INVENTION

It is the object of the invention to address the underlying lateral anatomical defect resulting in pelvic organ prolapse.

Another object of the invention is to provide a pessary or device that simultaneously addresses multiple pelvic organ defects.

Another object of the invention is to effectively support the bladder neck and prevent urinary incontinence.

Another object of this invention is to provide a pessary or device that is easy to insert and remove and allows patients to self insert and remove the pessary or device.

Another object of this invention is to make a pessary with a softer feel so that it is more comfortable for patients.

### SUMMARY OF THE INVENTION

This invention provides a device for treating or ameliorating a medical condition, comprising a base member having a first side opposite a second side and a third side opposite a fourth side, said base member defining a first plane, said first, second, third and fourth side defining a periphery of said base member, said third and fourth side being wider than said first and second side, said third and fourth side outlined with a ridge, a first lateral member opposite a second lateral member, said first and second lateral member extending outwardly respectively from said first and second side in a second plane intersecting said first plane, and a protruding member extending outwardly generally from a mid-section of said base member.

This invention provides a device for treating or ameliorating a medical condition, comprising a base member having a first side opposite a second side and a third side opposite a fourth side, said base member defining a first plane, said first, second, third and fourth side defining a periphery of the base member, said third and fourth side being wider than said first and second side, and said third and fourth side outlined with a ridge, and a protruding member extending outwardly generally from a mid-section of said base member, wherein perforations are provided on said base member for facilitating the flow of bodily fluid, wherein said base member comprises a convex and a concaved surface, wherein said protruding member extends outwardly from said convex surface.

This invention provides a device for treating, diagnosing, or ameliorating a medical condition in a subject, comprising an elongated or rectangularly shaped concaved body defining a first plane, two opposite side arms extending from two opposing periphery of said body in a second plane intersecting said first plane, said opposing periphery being the shorter sides of said body, and a handle extending outwardly from a convex surface of said body, wherein perforations is provided on said body, wherein said body further comprises a concaved surface.

An associated method for using the device of the present invention to treat, diagnose, or ameliorate various degrees of pelvic organ prolapse and/or urinary incontinence is also disclosed.

### DETAILED DESCRIPTION OF THE FIGURES

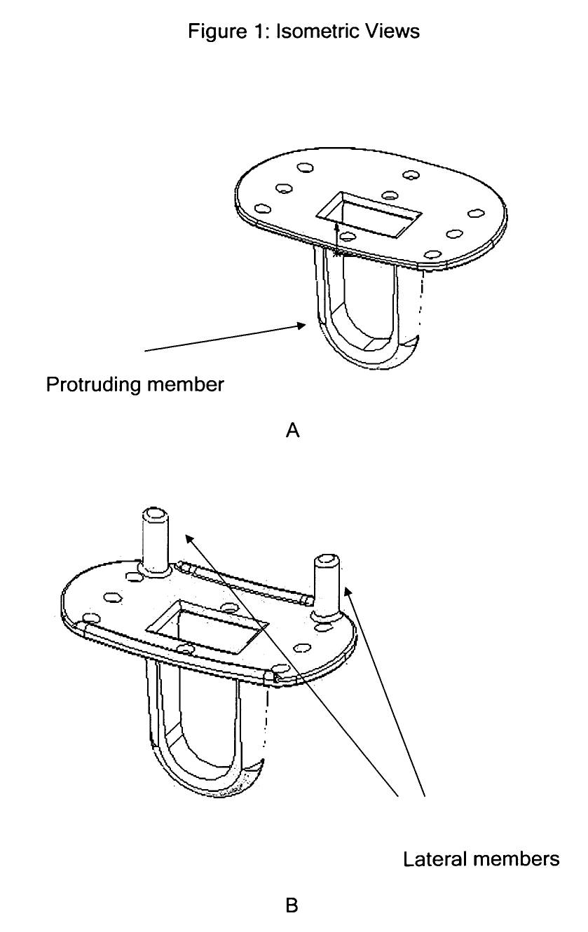

FIG. 1 shows isometric views of the device of the invention.

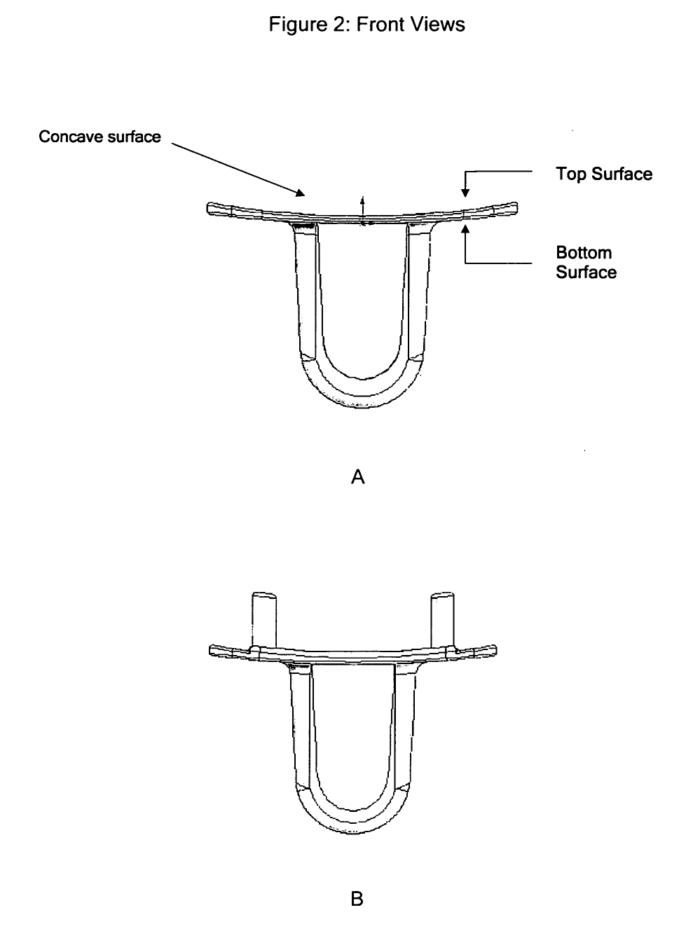

FIG. 2 shows a front view of the device of the invention.

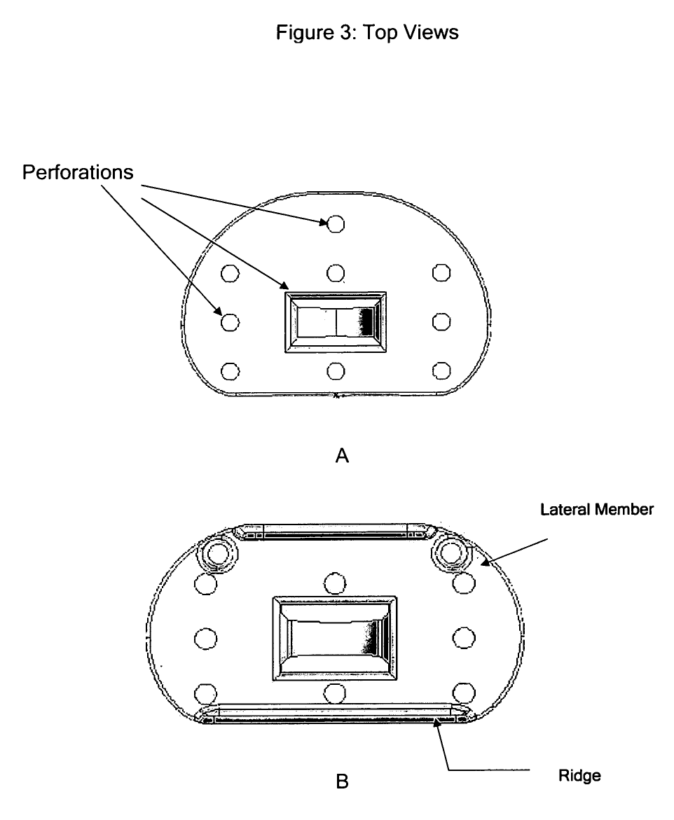

FIG. 3 shows a top view of the device of the invention.

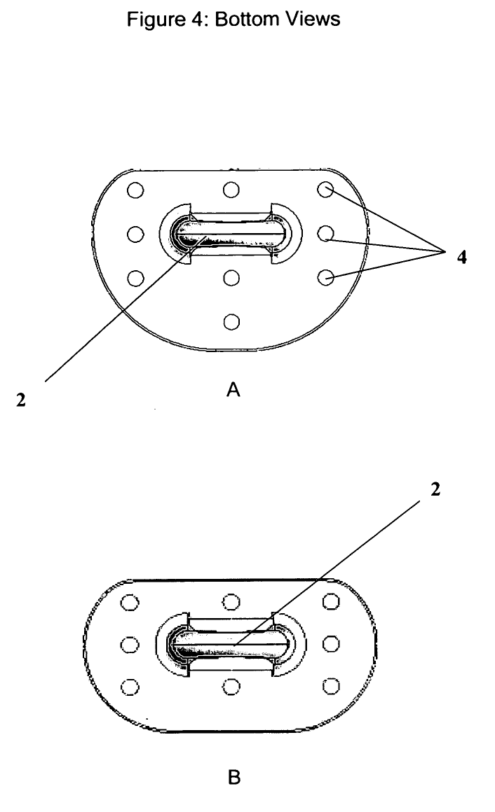

FIG. 4 shows a bottom view of the device of the invention.

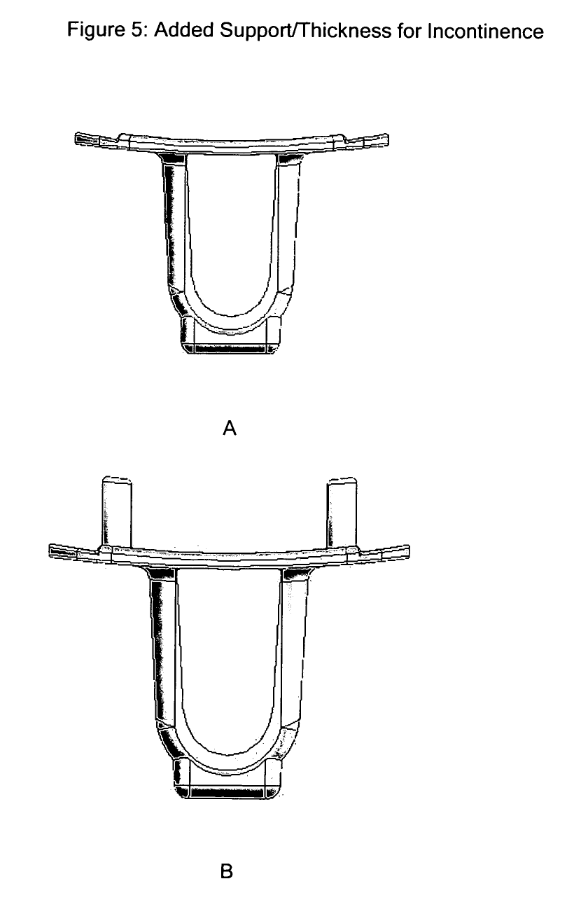

FIG. 5 shows a device of the invention with a protruding member or handle with added support for relieving urinary incontinence.

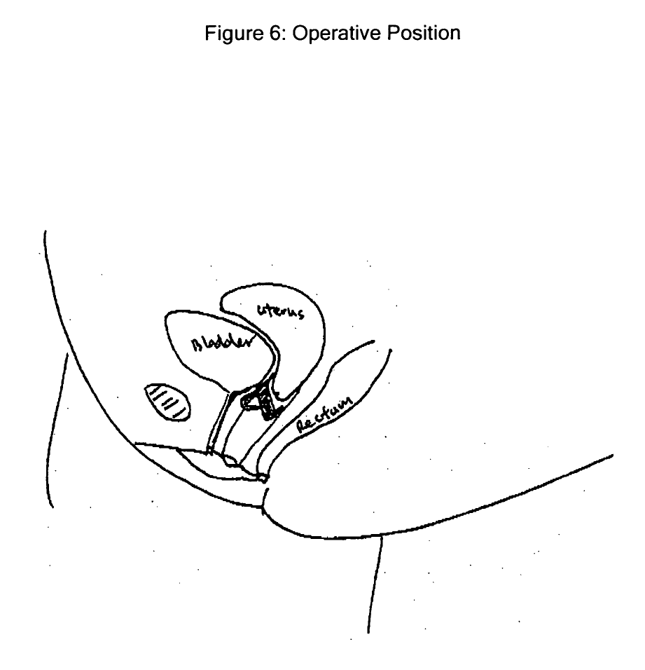

FIG. 6 shows a device of the invention in an operative position.

The present invention will be described in connection with preferred embodiments, however, it will be understood that this is no intent to limit the invention to the embodiments described. On the contrary, the intent is to cover all alternatives, modifications, and equivalents as may be included within the spirit and scope of the invention as defined by the appended claims.

### DETAILED DESCRIPTION OF THE INVENTION

The invention provides a pessary for treating multiple pelvic prolapses, vagina, bladder, rectum resulting from detachment of the endopelvic fascia from the pelvic side wall. This attachment site is called the Arcus Tendineus Levator Ani. This pessary has three main parts: two side arms, an elongated parabolic shaped body, and a handle. The two side arms once inserted into the vaginal cavity pushes the endopelvic fascia toward the ishial spines, which is the attachment point of the Arcus Tendineus Levator Ani. The body, is concaved toward the vaginal apex and the cervix, provide support for prolapse of the uterus and reducing herniation of small intestines (enteroceles). Having the concavity in the body helps create a suction and locks the pessary in place. The handle is attached to the convex side of the body, and angulated upward, to provide support to the bladder neck to prevent stress urinary incontinence, and to provide grasping means for easy removal of the pessary. In an embodiment, the handle comprises a hollow portion for easy grasping and handling of pessary within the vaginal cavity.

This invention provides a device for vaginal insertion that corrects the anatomical defect which is the cause of majority of prolapses. Said device comprises a base member (or body), two lateral members (or two opposite side arms) attached to two opposing sides of said base member (or body), and a protruding member (or handle) attached to the mid-section of said base member (or body) . In an embodiment, two lateral arms are provided to re-approximate the detached endopelvic fascia to the pelvic side walls at the site of the Arcus Tendineus Levator Ani. In another embodiment, the base member is elongated or rectangularly shaped, and is concaved toward the vaginal apex. In a further embodiment, the base member is adapted to reduce or to support the prolapsed uterus. The protruding member extends outwardly from the base member to support the bladder neck, thus improves urinary incontinence.

In a further embodiment, the device is formed of a biocompatible, flexible material, e.g., medical grade silicone, so that it can be folded at a mid-section for convenient insertion or removal.

This invention provides a device for treating or ameliorating a medical condition, comprising a base member having a first side opposite a second side and a third side opposite a fourth side, said base member defining a first plane, said first, second, third and fourth side defining a periphery of said base member, said third and fourth side being wider than said first and second side, said third and fourth side outlined with a ridge, a first lateral member opposite a second lateral member, said first and second lateral member extending outwardly respectively from said first and second side in a second plane intersecting said first plane, and a protruding member extending outwardly generally from a mid-section of said base member. In an embodiment, the medical condition selected from a group consisting of pelvic organ prolapse and urinary incontinence. In another embodiment, the base member, the first and second lateral member and the protruding member are respectively configured, adapted or shaped to re-approximate detached enoplevic fascia to pelvic side walls, to support prolapsed uterus, and to support the bladder neck in a subject. In another embodiment, the subject is a human subject. In a further embodiment, the subject is a primate.

In an embodiment of the invention, at least one perforation, opening or aperture is provided on the base member. In another embodiment, the opening is sufficiently large and numerous to permit bodily fluid to flow through the opening.

In an embodiment of the invention, the first and second side are generally parallel to each other, and the third and fourth side are generally parallel to each other. In another embodiment, the first and second sides have the same length, and the third and fourth side have the same length.

In an embodiment of the invention, the base member is parabolic or an elongated shape. In a further embodiment, the base member has a substantially parabolic concave shape. In a further embodiment, the elongated or rectangular base member includes a convex surface and a concave surface. In a further embodiment, the base member is concaved toward vaginal apex or cervix, or shaped to accommodate vaginal apex. In a further embodiment, the base member is configured or shaped to produce suction with vaginal apex. In a further embodiment, the base member is configured to concave toward the vaginal apex and the cervix for supporting prolapsed uterus and for reducing herniation of small intestines. The shape, curvature, fitting, size, and thickness of the base member appropriate for a particular patient will be apparent to one of ordinary skill in the art following the disclosure of this invention.

In an embodiment of the invention, the first and second lateral members are positioned respectively generally at a lower portion of the first and second side of the base member. In another embodiment, the first and second lateral members are positioned respectively along the first and second side of the base member. In a further embodiment, the first and second lateral members are positioned respectively generally at a mid-portion of the first and second side of the base member. The placement of the lateral members along the shorter sides of the base member is determined by the location and degree or severity of the prolapse along the pelvic side wall.

In an embodiment of the invention, the position, shape, orientation, size, rigidity, length, and/or dimension of the first and second lateral member, and/or the angulation of the first and second lateral member relative to the base member is determined by the location and degree or severity of the pelvic organ prolapse and/or urinary incontinence in a subject, and the configuration or fitting appropriate for a particular patient will be readily apparent to one of ordinary skill in the art based on the disclosure of this application.

In a further embodiment, the first and second lateral member are configured or formed to push the endopelvic fascia toward ishial spines. In a further embodiment, the angulation of the first and second lateral member relative to said base member is 120 degrees.

In an embodiment of the invention, the protruding member is tongue-shaped or U-shaped. In another embodiment, the length of the protruding member is dependent on the vaginal length of a subject. In a further embodiment, the protruding member extends outwardly from a convex surface of the base member. In a further embodiment, the angulation of the protruding member relative to said base member is 30 degrees. In a further embodiment, the thickness, length, rigidity, size, shape, orientation, and/or position of the protruding member is determined by the condition or severity of stress urinary incontinence in a subject.

In an embodiment of the invention, the base member, the first and second lateral member and the protruding member are appropriately configured, adapted, or shaped for facilitating insertion or removal of the device through the vaginal introitus of a subject.

This invention provides a device as described above without the two lateral members for treating, diagnosing, or ameliorating a minor form of pelvic organ prolapse in patients without lateral detachments.

This invention provides a device as described above comprising at least one lateral member for supporting for treating, diagnosing, or ameliorating a minor form of pelvic organ prolapse in patients without lateral detachments.

In an embodiment of the invention, the devices as described above are formed of a biocompatible, resilient material. In another embodiment, the biocompatible material includes silicone, rubber, polyvinyl chloride, elastomeric silicone, medical grade silicone, polyethylene, polypropylene, polyester, polyurethane, polyisobutylene, polychloroprene, and polybutadiene. In a further embodiment, the devices are described above are formed of a medical grade silicone.

In an embodiment of the invention, the base member, the first and second lateral member and the protruding member are formed as a unitary structure from medical grade silicone. In another embodiment, the base member, the first and second lateral member and the protruding member are formed as a single integral molded structure from a polymer, such as a resilient, biocompatible polymer or a medical grade silicone.

In an embodiment of the invention, the first and second lateral member are displaceable or bendable in an axis parallel to the third and fourth side of the base member into an elongated configuration for facilitating the insertion and removal of the device through the introitus of a subject. In another embodiment, the first and second lateral member are displaceable or bendable in an axis parallel to the third and fourth side of the base member into an elongated configuration for facilitating the insertion and removal of the device through the introitus of a subject.

In an embodiment of the invention, the protruding member is displaceable or bendable in an axis parallel to the first and second side of the base member into a compact configuration for facilitating the insertion and removal of the device through the introitus of a subject. In another embodiment, the protruding member is displaceable or bendable in an axis parallel to the third and fourth side of the base member into a compact configuration for facilitating the insertion and removal of the device through the introitus of a subject.

In an embodiment of the invention, the first and second lateral member and the protruding member includes a spring bias or memory tending to restore the first and second lateral member and the protruding member to the original, unfolded position, or to the deployed position.

In an embodiment of the invention, the base member is bendable or foldable along a fold axis located at the mid-section of the base member. The fold axis can be generally parallel to the first and second side of the base member or generally parallel to the third and fourth side of the base member.

This invention provides a device for treating, diagnosing, or ameliorating a medical condition in a subject, comprising an elongated or rectangularly shaped concaved body defining a first plane, two opposite side arms extending from two opposing periphery of said body in a second plane intersecting said first plane, said opposing periphery being the shorter sides of said body, and a handle extending outwardly from a convex surface of said body, wherein perforations is provided on said body, and wherein said body further comprises a concaved surface.

In an embodiment of the invention, the devices as described above are provided with a biocompatible, soft lining for reducing or eliminating discomfort to the wearer. In another embodiment, the devices as describe above are properly configured so that it does not move out of position during normal use. In a further embodiment, the devices as describe above is constructed and configured to maintain structural rigidity during normal use after being inserted into the vaginal cavity.

This invention provides a method for diagnosing the cause of pelvic organ prolapse, comprising the steps of inserting a device as described above into the vaginal cavity of a subject suffering from pelvic organ prolapse, positioning or placing the device in the vaginal cavity properly, and observing the cause of pelvic organ prolapse. The method can be used for pre-surgical examination to assess the degree or location of pelvic organ prolapse. In an embodiment, the above described method further comprises adjusting or orienting the device to treat or alleviate pelvic organ prolapse.

This invention provides a method for treating or ameliorating a medical condition in a subject, comprising the steps of (a) providing a device as described above; (b) folding or displacing the first and second lateral member and the protruding member closely against the base member to form an elongated compact form of the device; (c) inserting the elongated compact form of the device through the vaginal introitus of a subject; (d) deploying or placing the device in the vaginal cavity of the subject; and (e) aligning the device properly. In an embodiment, the medical condition is pelvic organ prolapse or urinary incontinence. In another embodiment, the first and second lateral member is configured or adapted to re-approximate detached endopelvic fascia to pelvic side walls at Arcus Tendineus Levator Ani, the base member is configured or adapted to reduce the prolapse of the uterus, and the protruding member is configured or adapted to support the bladder neck to alleviate urinary incontinence.

Other advantages and aspects of the present invention will become apparent upon reading the following examples.

### EXEMPLIFICATION

The invention being generally described, will be more readily understood by reference to the following examples which are included merely for purposes of illustration of certain aspects and embodiments of the present invention, and are not intended to limit the invention.

### EXAMPLES

For illustrative purposes only, several select embodiments are described. These are merely examples of the devices embodied by the invention, and should not limit the scope of the claimed invention. Referring to the drawings, FIG. 1 provides shows isometric views of embodiments of the device of the invention. FIGS. 1(A)and (B) show embodiments lacking or possessing lateral side arm members,respectively. Note that the base member 1 (or body) possesses perforations (or openings, slits, etc) as indicated in FIG. 4. From a surface of the base member there is a protruding member 2 (or handle) extending outwardly from the base member 1. FIGS. 1(C) and 1(D) shows two lateral members 5 attached to the base member 1 extending outwardly away from the base member 1. Due to the flexible and resilient material used in construction the device, the lateral member, when inserted, may extend from various angles, and not simply as illustrated.

FIG. 2 shows a front view of two embodiments of the device of the invention, illustrating the top and bottom surfaces. FIGS. 2(A) and 2(B) show the protruding member that may be used for gripping and moving the device. In some embodiments, the protruding member may also be used as a support member for pelvic organs. The member may be a tab or loop with a hollow center for ease of grasping and removal by the user, patient or a medical doctor. From this aspect, the concavity of the top surface or face is discernible.

FIG. 3 shows a top view of the surface opposite to that having the protruding member. FIG. 3 (B) shows an embodiment having both the elongated ridges and lateral members, whereas FIG. 3 (A) shows a similar surface without these structures. One purpose of the ridges is to diminish the erosion of the cervical and vaginal epithelial wall. This is accomplished by spreading the stabilizing contact force over a larger distance of the epithelial wall than when compared with the localized force exerted by the base member's rim. It should be clear that the device of the invention also encompasses embodiments where the profile in FIG. 3 (A) comprises the ridges, the lateral members, or both.

FIG. 4 is a bottom view that illustrates two embodiments of the device. From this view, the lateral members and/or ridges on the top surface are not visible.

FIG. 5 shows embodiments wherein the protruding member has added thickness 10 to diminish weakening when in contact with body fluids for long times. Such embodiments may be useful for treating cases of urinary incontinence or other conditions characterized by fluid in the vagina and/or cervix. To this end, the device may be fabricated of water-resistant polymers that also impart to the device the desired degree of flexibility and resilience. In addition, these embodiments may have increased numbers of openings, e.g., perforations, or slits, that allow the flow of bodily fluids. The side arms or lateral members are shown protruding from the base member.

FIG. 6 shows a device of the invention in the operative position within the vaginal cavity. As illustrated, the protruding loop member may also be fabricated of resilient material, so as to contribute to supporting the surrounding epithelial wall.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A device for treating or ameliorating a medical condition, comprising:
a base member having a first side opposite a second side and a third side opposite a fourth side, said base member defining a first plane, said first, second, third and fourth side defining a periphery of said base member, said third and fourth side being wider than said first and second side,
a first lateral member opposite a second lateral member, said first and second lateral member extending outwardly respectively from said first and second side in a second plane intersecting said first plane, and
a protruding member extending outwardly generally from a mid-section of said base member.

2. The device of claim 1, wherein said base member, said first and second lateral member and said protruding member are respectively configured to re-approximate detached enoplevic fascia to pelvic side walls, to support prolapsed uterus, and to support the bladder neck in a subject.

3. (canceled)

4. (canceled)

5. (canceled)

6. The device of claim 1, wherein one or more perforations are on said base member.

7. The device of claim 1, wherein said perforations are sufficiently large to permit bodily fluid to flow through said perforations.

8. The device of claim 1, wherein said third and fourth side are outlined with a ridge.

9. The device of claim 1, wherein said base member comprises a generally elongated shape configured to produce suction with the vaginal apex.

10. The device of claim 1, wherein said base member has a generally parabolic concave shape, concaved toward the vaginal apex or cervix, or configured to accommodate the vaginal apex.

11. The device of claim 1, wherein said based member comprises a generally concaved surface.

12. (canceled)

13. (canceled)

14. The device of claim 6, wherein a perforation is located generally at the center of the base member for breaking suction with vaginal apex for removing said device from a subject.

15. (canceled)

16. The device of claim 1, wherein said first and second lateral member are positioned respectively generally along said first and second side.

17. (canceled)

18. (canceled)

19. The device of claim 1, wherein the size, length, and dimension of said first and second lateral member, and the angulation of said first and second lateral member relative to said base member are dependent on a degree of pelvic organ prolapse or lateral detachment in a subject.

20. (canceled)

21. The device of claim 1, wherein the shape, size, length, and dimension of said protruding member, and the angulation of said protruding member relative to said base member are dependent on an amount of support needed for bladder neck in a subject suffering from urinary incontinence.

22. The device of claim 1, wherein said protruding member is tongue shaped or U-shaped, forming a loop or tab for grasping.

23. The device of claim 1, wherein the length of said protruding member is dependent on vaginal length of a subject.

24. The device of claim 1, wherein said protruding member extends outwardly from a convex surface of said base member.

25. The device of claim 1, wherein the length of said third and fourth side is outlined with a ridge.

26. (canceled)

27. The device of claim 1, wherein said base member, said first and second lateral member and said protruding member are appropriately configured for facilitating insertion or removal of said device through the vaginal introitus of a subject.

28. The device of claim 1, wherein said first and second lateral member is configured to push an endopelvic fascia toward ishial spines.

29. The device of claim 1, wherein said base member is configured to concave toward a vaginal apex and a cervix for providing support for prolapse of a uterus and for reducing herniation of small intestines.

30. (canceled)

31. The device of claim 1, wherein said device is formed of a biocompatible, resilient material.

32. The device of claim 31, wherein said biocompatible material is selected from the group including silicone, rubber, polyvinyl chloride, elastomeric silicone, medical grade silicone, polyethylene, polypropylene, polyester, polyurethane, polyisobutylene, polychloroprene, or polybutadiene.

33. The device of claim 31, wherein said device is formed of medical grade silicone.

34. (canceled)

35. (canceled)

36. (canceled)

37. The device of claim 33, wherein said polymer is medical grade silicone.

38. The device of claim 31, wherein said device is fabricated such that said first and second lateral member being displaceable or bendable in a first axis parallel to said third and fourth side, and that said protruding member being displaceable or bendable in a second axis parallel to said first and second side into an elongated configuration for facilitating insertion and removal of said device through a introitus of a subject, said first and second lateral member and said protruding member having a spring bias or memory tending to restore said first and second lateral member and said protruding member to an deployed position.

39. The device of claim 31, wherein said device is fabricated such that said protruding member being displaceable or bendable in an axis parallel to said third and fourth side into an elongated configuration for facilitating insertion and removal of said device through a introitus of a subject, said protruding member having a spring bias or memory tending to restore said protruding member to an deployed position.

40. The device of claim 31, wherein said device is fabricated such that said protruding member being displaceable or bendable in an axis parallel to said third and fourth side into an elongated configuration for facilitating insertion and removal of said device through a introits of a subject, said protruding member having a spring bias or memory tending to restore said protruding member to an deployed position.

41. (canceled)

42. (canceled)

43. (canceled)

44. (canceled)

45. (canceled)

46. The device of claim 31, wherein said device is suitable for treating a disease or condition selected from the group consisting of pelvic organ prolapse and urinary incontinence.

47. (canceled)

48. (canceled)

49. (canceled)

50. A method for diagnosing the cause of pelvic organ prolapse, comprising the steps of:
inserting the device of claim 1 or 31 into the vaginal cavity of a subject suffering from pelvic organ prolapse,
positioning said device in the vaginal cavity appropriately, and
observing the exact cause of pelvic organ prolapse.

51. (canceled)

52. (canceled)

53. A method for treating or ameliorating a medical condition in a subject, comprising the steps of:
(a) providing the device of claim 1 or 31;
(b) folding or displacing said first and second lateral member and said protruding member closely against said base member to form an elongated compact form of said device;
(c) inserting said elongated compact form of said device through the introitus of said subject;
(d) deploying said device in vaginal cavity of said subject; and
(e) aligning said device for proper operation,
wherein said medical condition is pelvic organ prolapse or urinary incontinence, and wherein said first and second lateral member and said protruding member having a spring bias or memory tending to restore said first and second lateral member and said protruding member to an deployed position.

54. (canceled)

55. (canceled)

56. (canceled)

57. (canceled)

58. (canceled)

59. (canceled)

60. (canceled)

61. The device of claim 1 or 31, wherein the thickness of said protruding member depends on the condition or severity of stress urinary incontinence in a subject.

62. The device of claim 1 or 31, wherein the thickness of the said handle depends on the condition or severity of stress urinary incontinence in a subject.
